(19) United States
(12) Patent Application Publication
(10) Pub. No.: US 2010/0168704 A1
(43) Pub. Date: Jul. 1, 2010

(54) METHOD OF FORMING AN ELASTIC LAMINATE INCLUDING A CROSS-LINKED ELASTIC FILM

(76) Inventors: Oomman P. Thomas, Alpharetta, GA (US); Jose Augusto Vidal de Siqueira, Roswell, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/347,434

(22) Filed: Dec. 31, 2008

(51) Int. Cl. A61F 13/56; B32B 37/12; B32B 27/32

(52) U.S. Cl. 604/366; 156/327; 156/273.3; 428/523

(57) ABSTRACT

A method of forming an elastic composite formed from a laminate that contains a cross-linked elastic film and a nonwoven facing is provided.

### BACKGROUND OF THE INVENTION

Elastic composites are commonly incorporated into products (e.g., diapers, training pants, garments, and so forth) to improve their ability to better fit the contours of the body. For example, the elastic composite may be formed from an elastic film and a nonwoven facing. In the current range of elastic materials available, there is a clear gap between medium and high performance elastic materials. For example, materials based on styrenic SEBS polymer generally provide medium performance material with relatively high stress relaxation compared to Spandex™ or Lycra™ based materials which have lower stress relaxation. However, Spandex™ and Lycra™ materials are much more expensive compared to styrenic block copolymers. Nonetheless, the better elastic performance demonstrated by the expensive elastic materials is desirable.

As such, a need currently exists for a cost-effective elastic composite that is formed from a lightweight and low strength nonwoven facing, yet also exhibits elastic performance nearing that of more expensive high performance elastics.

### SUMMARY OF THE INVENTION

In accordance with one embodiment of the present invention, a method of forming an elastic composite is disclosed. The method includes extruding an elastomeric core layer composition and an optional first skin layer composition directly onto a surface of a first nonwoven facing to form a first film/nonwoven laminate characterized by the elastic core layer bonded either directly to the first nonwoven facing or bonded indirectly to the first nonwoven facing by the intervening optional first skin layer. Following preparation of the first film/nonwoven laminate, the exposed elastomeric core layer composition is cross-linked to improve elastic performance. A second facing layer may then be laminated to the exposed elastomeric core layer of the first film/nonwoven laminate. The second facing layer may include a nonwoven facing layer and, optionally, a film layer. The optional film layer may include a skin layer or core layer as in the first film/nonwoven laminate. The exposed cross-linked core layer of the first film/nonwoven laminate and the exposed second facing layer are positioned in face-to-face relation. The first and second film/nonwoven laminates are then bonded together.

In accordance with another embodiment, another method of forming an elastic composite is disclosed. The method includes the steps of extruding a thermoplastic composition directly onto a surface of a first nonwoven to form a first film, wherein the thermoplastic composition comprises a cross-linkable elastic polymer; allowing the first film to bond to the first nonwoven to form a laminate; cross-linking the cross-linkable elastic polymer; and thereafter joining the first film directly to a second facing, the second facing comprising a second nonwoven. In one aspect, the second facing includes a second film, wherein the first film is positioned and bonded directly to the second film.

In one embodiment, the first nonwoven may have a basis weight of about 45 grams per square meter or less and a peak load of about 350 grams-force per inch or less in the cross-machine direction. In another embodiment, the first nonwoven may be a meltblown web. In a further embodiment, the first nonwoven may include a polyethylene polymer.

In one embodiment, the first film includes an elastic layer and a thermoplastic layer, wherein the thermoplastic layer is positioned between the first nonwoven and the elastic layer. In another embodiment, the elastic layer includes the cross-linkable elastic polymer.

In another embodiment, the second facing may include a polypropylene polymer.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 schematically illustrates a method for forming a composite according to one embodiment of the present invention; and

FIG. 2 is a cross-sectional illustration of one embodiment of the composite of the present invention.

Repeat use of reference characters in the present specification and drawings is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

Generally speaking, the present invention is directed to an elastic composite formed from a laminate that contains a crosslinked elastic film and a lightweight nonwoven facing desirably having a low strength in the cross-machine direction (“CD”). The “cross-machine direction” or “CD” is the direction perpendicular to the direction in which a material is produced. The “machine direction” is the direction in which a material is produced. Due to the low strength of the facing, it is desirable that the elastic film have a sufficient thickness and weight to enhance the strength of the resulting composite. In this regard, a second facing may be employed in the elastic composite that is formed from a nonwoven facing and an optional thermoplastic film to impart increased strength to the composite. The optional film and facing of the second facing may be formed from the same or different materials than the film and facing of the first laminate. Regardless, the laminates are positioned so that the elastic and thermoplastic films are in a face-to-face relationship in the final composite. Prior to placing the film/nonwoven laminate and second facing material in the face-to-face relationship, the elastic core film is cross-linked. Cross-linking the elastic core prior to placing the laminates in the face-to-face relationship results in more efficient cross-linking in the elastic core and reduces exposure of the other components in the laminate to the cross-linking process. After cross-linking, and after the film/nonwoven laminate and the second facing material are positioned in the face-to-face relationship, the materials may be readily joined together under light pressure, under conventional calendar bonding processes, through use of adhesives, through grooved rolling, and so forth.

In this regard, various embodiments of the present invention will now be described in more detail.

### I. First Laminate

A. Nonwoven Facing

As stated above, the nonwoven facing of the first laminate is generally lightweight and has a low degree of strength in the cross-machine direction (“CD”), which increases the flexibility of the composite and also provides significant costs savings in its manufacture. More specifically, the basis weight may range from about 45 grams per square meter or less, in some embodiments from about 1 to about 30 grams per square meter, and in some embodiments, from about 2 to about 20 grams per square meter. Likewise, the nonwoven facing may have a peak load in the cross-machine direction of about 350 grams-force per inch (width) or less, in some embodiments about 300 grams-force per inch or less, in some embodiments from about 50 to about 300 grams-force per inch, in some embodiments from about 60 to about 250 grams-force per inch, and in some embodiments, from about 75 to about 200 grams-force per inch. If desired, the nonwoven facing may also have a low strength in the machine direction (“MD”), such as a peak load in the machine direction of about 3000 grams-force per inch (width) or less, in some embodiments about 2500 grams-force per inch or less, in some embodiments from about 50 to about 2000 grams-force per inch, and in some embodiments, from about 100 to about 1500 grams-force per inch.

The nonwoven facing is generally a web having a structure of individual fibers or threads which are interlaid, but not in an identifiable manner as in a knitted fabric. Examples of suitable nonwoven facings include, but are not limited to, meltblown webs, spunbond webs, bonded carded webs, airlaid webs, coform webs, hydraulically entangled webs, combinations of the foregoing, and so forth. The nonwoven facing may be formed from a variety of known processes, such as meltblowing, spunbonding, carding, wet laying, air-laying, hydro-entangling, coform, and so forth.

Meltblown webs or facings are nonwoven webs that are formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto for all purposes. In one particular embodiment, for example, the nonwoven facing is a meltblown facing that contains “microfibers” in that they have an average size of about 15 micrometers or less, in some embodiments from about 0.01 to about 10 micrometers, and in some embodiments, from about 0.1 to about 5 micrometers.

Spunbond webs or facings are nonwoven webs containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers often have a diameter of from about 10 to about 20 micrometers.

Polymers that may be used to form nonwoven facings may include, for instance, polyolefins, e.g., polyethylene, polypropylene, polybutylene, and so forth; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers thereof; blends thereof; and so forth. In one embodiment, the nonwoven facing includes a polyethylene polymer that will cross-link upon exposure to electromagnetic radiation. It should be noted that the polymer(s) may also contain other additives, such as processing aids or treatment compositions to impart desired properties to the fibers, residual amounts of solvents, pigments or colorants, and so forth.

Monocomponent and/or multicomponent fibers may be used to form the nonwoven facing. Various methods for forming multicomponent fibers are described in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference thereto for all purposes. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference thereto for all purposes.

The desired denier of the fibers used to form the nonwoven facing may vary depending on the desired application. Typically, the fibers are formed to have a denier per filament (i.e., the unit of linear density equal to the mass in grams per 9000 meters of fiber) of less than about 6, in some embodiments less than about 3, and in some embodiments, from about 0.5 to about 3.

Although not required, the nonwoven facing may be optionally bonded using any conventional technique, such as with an adhesive or autogenously (e.g., fusion and/or self-adhesion of the fibers without an applied external adhesive). Suitable autogenous bonding techniques may include ultrasonic bonding, thermal bonding, through-air bonding, calender bonding, and so forth. The temperature and pressure required may vary depending upon many factors including but not limited to, pattern bond area, polymer properties, fiber properties and nonwoven properties. For example, the facing may be passed through a nip formed between two rolls, both of which are typically not patterned i.e., smooth. In this manner, only a small amount of pressure is exerted on the materials to lightly bond them together. Without intending to be limited by theory, the present inventors believe that such lightly bonded materials can retain a higher degree of extensibility and thereby increase the elasticity and extensibility of the resulting composite. For example, the nip pressure may range from about 0.1 to about 20 pounds per linear inch, in some embodiments from about 1 to about 15 pounds per linear inch, and in some embodiments, from about 2 to about 10 pounds per linear inch. One or more of the rolls may likewise have a surface temperature of from about 15° C. to about 60° C., in some embodiments from about 20° C. to about 50° C., and in some embodiments, from about 25° C. to about 40° C.

The nonwoven facing may also be stretched in the machine and/or cross-machine directions prior to lamination to the film of the present invention. Suitable stretching techniques may include necking, tentering, groove roll stretching, and so forth. For example, the facing may be necked such as described in U.S. Pat. Nos. 5,336,545, 5,226,992, 4,981,747 and 4,965,122 to Morman, as well as U.S. Patent Application Publication No. 2004/0121687 to Morman, et al. Alternatively, the nonwoven facing may remain relatively inextensible in at least one direction prior to lamination to the film. In such embodiments, the nonwoven facing may be optionally stretched in one or more directions subsequent to lamination to the film. The facing may also be subjected to other known processing steps, such as aperturing, heat treatments, and so forth.

B. Elastic Film

The elastic film of the first laminate is formed from one or more elastomeric polymers that are melt-processable, i.e., thermoplastic. Any of a variety of thermoplastic elastomeric polymers may generally be employed in the present invention, such as elastomeric polyesters, elastomeric polyurethanes, elastomeric polyamides, elastomeric copolymers, elastomeric polyolefins, and so forth. In one embodiment, for instance, a substantially amorphous block copolymer may be employed that contains blocks of a monoalkenyl arene and a saturated conjugated diene. Such block copolymers are particularly useful in the present invention due to their high degree of elasticity and tackiness, which enhances the ability of the film to bond to the nonwoven facing.

The monoalkenyl arene block(s) may include styrene and its analogues and homologues, such as o-methyl styrene; p-methyl styrene; p-tert-butyl styrene; 1,3 dimethyl styrene p-methyl styrene; and so forth, as well as other monoalkenyl polycyclic aromatic compounds, such as vinyl naphthalene; vinyl anthrycene; and so forth. Preferred monoalkenyl arenes are styrene and p-methyl styrene. The conjugated diene block(s) may include homopolymers of conjugated diene monomers, copolymers of two or more conjugated dienes, and copolymers of one or more of the dienes with another monomer in which the blocks are predominantly conjugated diene units. Preferably, the conjugated dienes contain from 4 to 8 carbon atoms, such as 1,3 butadiene (butadiene); 2-methyl-1,3 butadiene; isoprene; 2,3 dimethyl-1,3 butadiene; 1,3 pentadiene (piperylene); 1,3 hexadiene; and so forth. The amount of monoalkenyl arene (e.g., polystyrene) blocks may vary, but typically constitute from about 8 wt. % to about 55 wt. %, in some embodiments from about 10 wt. % to about 35 wt. %, and in some embodiments, from about 15 wt. % to about 25 wt. % of the copolymer. Suitable block copolymers may contain monoalkenyl arene endblocks having a number average molecular weight from about 5,000 to about 35,000 and saturated conjugated diene midblocks having a number average molecular weight from about 20,000 to about 170,000. The total number average molecular weight of the block polymer may be from about 30,000 to about 250,000.

Particularly suitable thermoplastic elastomeric copolymers are available from Kraton Polymers LLC of Houston, Tex. under the trade name KRATON®. KRATON® polymers include styrene-diene block copolymers, such as styrene-butadiene, styrene-isoprene, styrene-butadiene-styrene, styrene-isoprene-styrene, mixtures thereof, and so forth. KRATON® polymers also include styrene-olefin block copolymers formed by selective hydrogenation of styrene-diene block copolymers. Examples of such styrene-olefin block copolymers include styrene-(ethylene-butylene), styrene-(ethylene-propylene), styrene-(ethylene-butylene)-styrene, styrene-(ethylene-propylene)-styrene, styrene-(ethylene-butylene)-styrene-(ethylene-butylene), styrene-(ethylene-propylene)-styrene-(ethylene-propylene), styrene-ethylene-(ethylene-propylene)-styrene, mixtures thereof, and so forth. These block copolymers may have a linear, radial or star-shaped molecular configuration. Specific KRATON® block copolymers include those sold under the brand names G 1652, G 1657, G 1730, MD6673, and MD6973. Various suitable styrenic block copolymers are described in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON®. Still other suitable copolymers include the S-I-S and S-B-S elastomeric copolymers available from Dexco Polymers of Houston, Tex. under the trade designation VECTOR®. Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

Of course, other thermoplastic elastomeric polymers may also be used to form the film, either alone or in conjunction with the block copolymers. Semi-crystalline polyolefins, for example, may be employed that have or are capable of exhibiting a substantially regular structure. Exemplary semi-crystalline polyolefins include polyethylene, polypropylene, blends and copolymers thereof. In one particular embodiment, a polyethylene is employed that is a copolymer of ethylene and an α-olefin, such as a C₃-C₂₀ α-olefin or C₃-C₁₂ α-olefin. Suitable α-olefins may be linear or branched (e.g., one or more C₁-C₃ alkyl branches, or an aryl group). Specific examples include 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Preferred polyethylenes for use in the present invention are ethylene-based copolymer plastomers available under the designation EXACT™ from ExxonMobil Chemical Company of Houston, Tex. Other suitable polyethylene plastomers are available under the designation ENGAGE™ and AFFINITY™ from Dow Chemical Company of Midland, Mich. Still other suitable ethylene polymers are available from The Dow Chemical Company under the designations DOWLEX™ (LLDPE) and ATTANE™ (ULDPE). Other suitable ethylene polymers are described in U.S. Pat. No. 4,937,299 to Ewen et al.; U.S. Pat. No. 5,218,071 to Tsutsui et al.; U.S. Pat. No. 5,272,236 to Lai, et al.; and U.S. Pat. No. 5,278,272 to Lai, et al., which are incorporated herein in their entirety by reference thereto for all purposes.

Of course, the present invention is by no means limited to the use of ethylene polymers. For instance, propylene plastomers may also be suitable for use in the film. Suitable plastomeric propylene polymers may include, for instance, copolymers or terpolymers of propylene include copolymers of propylene with an α-olefin (e.g., C₃-C₂₀), such as ethylene, 1-butene, 2-butene, the various pentene isomers, 1-hexene, 1-octene, 1-nonene, 1-decene, 1-unidecene, 1-dodecene, 4-methyl-1-pentene, 4-methyl-1-hexene, 5-methyl-1-hexene, vinylcyclohexene, styrene, and so forth. Suitable propylene polymers are commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. No. 6,500,563 to Datta, et al.; U.S. Pat. No. 5,539,056 to Yang, et al.; and U.S. Pat. No. 5,596,052 to Resconi, et al., which are incorporated herein in their entirety by reference thereto for all purposes.

Any of a variety of known techniques may generally be employed to form the semi-crystalline polyolefins. For instance, olefin polymers may be formed using a free radical or a coordination catalyst (e.g., Ziegler-Natta). Preferably, the olefin polymer is formed from a single-site coordination catalyst, such as a metallocene catalyst.

Of course, besides elastomeric polymers, generally inelastic thermoplastic polymers may also be used so long as they do not adversely affect the elasticity of the composite. For example, the thermoplastic composition may contain other polyolefins (e.g., polypropylene, polyethylene, and so forth.). In one embodiment, the thermoplastic composition may contain an additional propylene polymer, such as homopolypropylene or a copolymer of propylene. The additional propylene polymer may, for instance, be formed from a substantially isotactic polypropylene homopolymer or a copolymer containing equal to or less than about 10 wt. % of other monomer, i.e., at least about 90% by weight propylene. Such a polypropylene may be present in the form of a graft, random, or block copolymer and may be predominantly crystalline in that it has a sharp melting point above about 110° C., in some embodiments about above 115° C., and in some embodiments, above about 130° C. Examples of such additional polypropylenes are described in U.S. Pat. No. 6,992,159 to Datta, et al., which is incorporated herein in its entirety by reference thereto for all purposes.

The elastic film may also contain other components as is known in the art. In one embodiment, for example, the elastic film contains a filler. Fillers are particulates or other forms of material that may be added to the film polymer extrusion blend and that will not chemically interfere with the extruded film, but which may be uniformly dispersed throughout the film. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways. Breathable microporous elastic films are described, for example, in U.S. Pat. Nos. 5,997,981; 6,015,764; and 6,111,163 to McCormack, et al.; U.S. Pat. No. 5,932,497 to Morman, et al.; U.S. Pat. No. 6,461,457 to Taylor, et al., which are incorporated herein in their entirety by reference thereto for all purposes. Examples of suitable fillers include, but are not limited to, calcium carbonate, various kinds of clay, silica, alumina, barium carbonate, sodium carbonate, magnesium carbonate, talc, barium sulfate, magnesium sulfate, aluminum sulfate, titanium dioxide, zeolites, cellulose-type powders, kaolin, mica, carbon, calcium oxide, magnesium oxide, aluminum hydroxide, pulp powder, wood powder, cellulose derivatives, chitin and chitin derivatives. In certain cases, the filler content of the film may range from about 25 wt. % to about 75 wt. %, in some embodiments, from about 30 wt. % to about 70 wt. %, and in some embodiments, from about 40 wt. % to about 60 wt. % of the film.

Other additives may also be incorporated into the film, such as melt stabilizers, crosslinking catalysts, pro-rad additives, processing stabilizers, heat stabilizers, light stabilizers, antioxidants, heat aging stabilizers, whitening agents, antiblocking agents, bonding agents, tackifiers, viscosity modifiers, and so forth. Examples of suitable tackifier resins may include, for instance, hydrogenated hydrocarbon resins. REGALREZ™ hydrocarbon resins are examples of such hydrogenated hydrocarbon resins, and are available from Eastman Chemical. Other tackifiers are available from ExxonMobil under the ESCOREZ™ designation. Viscosity modifiers may also be employed, such as polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganoxe”, such as Irganox® 1076, 1010, or E 201. Moreover, bonding agents may also be added to the film to facilitate bonding of the film to additional materials (e.g., nonwoven web). Typically, such additives (e.g., tackifier, antioxidant, stabilizer, and so forth) are each present in an amount from about 0.001 wt. % to about 25 wt. %, in some embodiments, from about 0.005 wt. % to about 20 wt. %, and in some embodiments, from 0.01 wt. % to about 15 wt. % of the film.

The elastic film of the present invention may be mono- or multi-layered. Multi-layered films may be prepared by co-extrusion or any other conventional layering technique. When employed, the multi-layered film typically contains at least one thermoplastic (or plastic) layer and at least one elastic layer. The thermoplastic layer may be employed to provide strength and integrity to the resulting composite, while the elastic layer may be employed to provide elasticity and sufficient tack for adhering to the nonwoven facing. To impart the desired elastic properties to the film, elastomers typically constitute about 55 wt. % or more, in some embodiments about 60 wt. % or more, and in some embodiments, from about 65 wt. % to 100 wt. % of the polymer content of the elastomeric composition used to form the elastic layer(s). In fact, in certain embodiments, the elastic layer(s) may be generally free of polymers that are inelastic. For example, such inelastic polymers may constitute about 15 wt. % or less in some embodiments about 10 wt. % or less, and in some embodiments, about 5 wt. % or less of the polymer content of the elastomeric composition.

Although the thermoplastic layer(s) may possess some degree of elasticity, such layers are generally formed from a thermoplastic composition that is less elastic than the elastic layer(s) to ensure that the strength of the film is sufficiently enhanced. For example, one or more elastic layers may be formed primarily from substantially amorphous elastomers (e.g., styrene-olefin copolymers) and one or more thermoplastic layers may be formed from polyolefin plastomers (e.g., single-site catalyzed ethylene or propylene copolymers), which are described in more detail above. Although possessing some elasticity, such polyolefins are generally less elastic than substantially amorphous elastomers. Of course, the thermoplastic layer(s) may contain generally inelastic polymers, such as conventional polyolefins, e.g., polyethylene (low density polyethylene (“LDPE”), Ziegler-Natta catalyzed linear low density polyethylene (“LLDPE”), and so forth), polypropylene, ethylene butene copolymer, polybutylene, and so forth; polytetrafluoroethylene; polyesters, e.g., polyethylene terephthalate, and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers and mixtures thereof; and so forth. In certain embodiments, polyolefins (e.g., conventional and/or plastomers) are employed and constitute about 55 wt. % or more, in some embodiments about 60 wt. % or more, and in some embodiments, from about 65 wt. % to 100 wt. % of the polymer content of the thermoplastic composition used to form the thermoplastic layer(s).

The thickness of the thermoplastic and elastic layers is generally selected so as to achieve the desired degree of film elasticity and strength. For instance, the thickness of an elastic layer is typically from about 20 to about 200 micrometers, in some embodiments from about 25 to about 175 micrometers, and in some embodiments, from about 30 to about 150 micrometers. The elastic layer(s) may also constitute from about 70% to about 99.5% of the total thickness of the film, and in some embodiments from about 80% to about 99% of the total thickness of the film. On the other hand, the thickness of a thermoplastic layer(s) is typically from about 0.5 to about 20 micrometers, in some embodiments from about 1 to about 15 micrometers, and in some embodiments, from about 2 to about 12 micrometers. The elastic layer(s) may also constitute from about 0.5% to about 30% of the total thickness of the film, and in some embodiments from about 1% to about 20% of the total thickness of the film. In some embodiments the film may have a total thickness (all layers combined) of from about 20 to about 250 micrometers, in some embodiments, from about 25 to about 225 micrometers, and in some embodiments, from about 30 to about 200 micrometers.

Following lamination to the nonwoven and prior to lamination to the second laminate, an elastomeric polymer employed in the film is crosslinked to provide the film with enhanced elastic characteristics. Crosslinking is generally achieved through the formation of free radicals (unpaired electrons) that link together to form a plurality of carbon-carbon covalent bonds. These bonds create a three-dimensional network from the original linear polymer chains. Upon crosslinking, the three-dimensional crosslinked network may provide the material with additional elasticity and/or improved hysteresis properties in the machine direction, cross-machine direction, or both.

Free radical formation is generally induced through electromagnetic radiation, either alone or in the presence of pro-rad additives. Some suitable examples of electromagnetic radiation that may be used include, but are not limited to, ultraviolet light electron beam radiation, natural and artificial radio isotopes (e.g., α, β, and γ rays), x-rays, neutron beams, positively-charged beams, laser beams, and so forth. Electron beam radiation, for instance, involves the production of accelerated electrons by an electron beam device. Electron beam devices are generally well known in the art. For instance, in one embodiment, an electron beam device may be used that is available from Energy Sciences, Inc., of Woburn, Mass. under the name “Microbeam LV.” Other examples of suitable electron beam devices are described in U.S. Pat. No. 5,003,178 to Livesay; U.S. Pat. No. 5,962,995 to Avnery; U.S. Pat. No. 6407492 to Avnery, et al., which are incorporated herein in their entirety by reference thereto for all purposes.

When supplying electromagnetic radiation, it is generally desired to selectively control various parameters of the radiation to enhance the degree of crosslinking of, for example, diene polymers, semi-crystalline polyolefin(s), and so forth. For example, one parameter that may be controlled is the wavelength λ of the electromagnetic radiation. Specifically, the wavelength λ of the electromagnetic radiation varies for different types of radiation of the electromagnetic radiation spectrum. Although not required, the wavelength λ of the electromagnetic radiation is generally about 1000 nanometers or less, in some embodiments about 100 nanometers or less, and in some embodiments, about 1 nanometer or less. Electron beam radiation, for instance, typically has a wavelength λ of about 1 nanometer or less. Besides selecting the particular wavelength λ of the electromagnetic radiation, other parameters may also be selected to achieve the desired degree of crosslinking. For example, higher accelerating voltage, dosage and energy levels of radiation will typically result in a higher degree of crosslinking; however, it is generally desired that the materials not be “overexposed” to radiation. Such overexposure may result in an unwanted level of product degradation. Thus, in some embodiments, the accelerating voltage may range from about 50 kV (kilovolts) to about 300 kV, and in other embodiments from about 75 kV to about 250 kV, and in further embodiments from about 100 kV to about 200 kV. Dosage may range from about 1 megarad (Mrad) to about 30 Mrads, in some embodiments, from about 3 Mrads to about 25 Mrads, and in other embodiments, from about 5 to about 15 Mrads. In addition, the energy level in some embodiments may range from about 0.05 megaelectron volts (MeV) to about 600 MeV.

It should be understood, however, that the actual dosage and/or energy level required depends on the type of polymers and electromagnetic radiation. Specifically, certain types of polymers may tend to form a lesser or greater number of crosslinks, which will influence the dosage and energy of the radiation utilized. Likewise, certain types of electromagnetic radiation may be less effective in crosslinking the polymer, and thus may be utilized at a higher dosage and/or energy level. For instance, electromagnetic radiation that has a relatively high wavelength (lower frequency) may be less efficient in crosslinking the polymer than electromagnetic radiation having a relatively low wavelength (higher frequency). Accordingly, in such instances, the desired dosage and/or energy level may be increased to achieve the desired degree of crosslinking.

### II. Second Facing/Laminate

A. Nonwoven Facing

The nonwoven facing of the second facing/laminate may include any suitable nonwoven material, such as a meltblown web, spunbond web, bonded carded web, wet-laid web, airlaid web, coform web, hydraulically entangled web, and so forth, as well as combinations of the foregoing. For examples, any of the nonwoven materials described above for the first nonwoven facing may be employed. In one particular embodiment, the facing may be a bonded carded facing. In another embodiment, the facing may include a polypropylene polymer. Fibers of any desired length may be employed in the bonded carding facing, such as staple fibers, continuous fibers, and so forth. For example, staple fibers may be used that have a fiber length in the range of from about 1 to about 150 millimeters, in some embodiments from about 5 to about 50 millimeters, in some embodiments from about 10 to about 40 millimeters, and in some embodiments, from about 10 to about 25 millimeters. Such fibers may be formed into a carded web by placing bales of the fibers into a picker that separates the fibers. Next, the fibers are sent through a combing or carding unit that further breaks apart and aligns the fibers in the machine direction so as to form a machine direction-oriented fibrous nonwoven web. The carded web may then be lightly bonded in a manner such as described above.

Although not required, the nonwoven of the second facing/laminate may also be lightweight and of low strength. For example, the basis weight of the nonwoven may range from about 1 to about 45 grams per square meter, in some embodiments from about 2 to about 30 grams per square meter, and in some embodiments, from about 3 to about 20 grams per square meter. The nonwoven may also have a peak load in the cross-machine direction (“CD”) of about 350 grams-force per inch (width) or less, in some embodiments about 300 grams-force per inch or less, in some embodiments from about 50 to about 300 grams-force per inch, in some embodiments from about 60 to about 250 grams-force per inch, and in some embodiments, from about 75 to about 200 grams-force per inch. If desired, the nonwoven facing may also have a low strength in the machine direction (“MD”), such as a peak load in the machine direction of about 3000 grams-force per inch (width) or less, in some embodiments about 2500 grams-force per inch or less, in some embodiments from about 50 to about 2000 grams-force per inch, and in some embodiments, from about 100 to about 1500 grams-force per inch.

As described above, the nonwoven facing of the second facing/laminate may also be stretched in the machine and/or cross-machine directions prior to lamination to the film of the present invention, as well as subjected to other known processing steps, such as aperturing, heat treatments, and so forth.

B. Optional Film

The optional thermoplastic film of the second facing/laminate is formed from one or more thermoplastic polymers. If desired, the thermoplastic polymers may be elastomers, such as described above, such that the film possesses a certain degree of elasticity. Such elastomers may be the same or different than the elastomers used in the elastic film of the first laminate. As described above for the first laminate, the optional film may be crosslinked after lamination to the second nonwoven and prior to lamination of the second facing/laminate to the first laminate. In one embodiment for example, an optional thermoplastic skin layer may be formed from a composition that includes substantially polyolefin plastomers (e.g., single-site catalyzed ethylene or propylene copolymers) and an elastic core may be formed from a composition that includes amorphous elastomers (e.g., styrene-olefin copolymers), which are described in more detail above. Of course, the thermoplastic film may also contain generally inelastic polymers as described above. In fact, polyolefins (e.g., conventional and/or plastomers) may constitute about 55 wt. % or more, in some embodiments about 60 wt. % or more, and in some embodiments, from about 65 wt. % to 100 wt. % of the polymer content of the composition used to form one or more layers of the thermoplastic film. The thermoplastic film may also have a mono-layered or multi-layered structure, such as described above.

Regardless of the content, the basis weight of the optional elastic core and the optional thermoplastic skin layer are generally selected so as to achieve an appropriate balance between film elasticity and strength. For instance, the basis weight of the elastic core may range from about 1 to about 45 grams per square meter, in some embodiments from about 2 to about 30 grams per square meter, and in some embodiments, from about 5 to about 20 grams per square meter. The basis weight of the thermoplastic skin layer may likewise range from about 1 to about 45 grams per square meter, in some embodiments from about 2 to about 30 grams per square meter, and in some embodiments, from about 5 to about 20 grams per square meter. The film may also have a total thickness of from about 1 to about 100 micrometers, in some embodiments, from about 10 to about 80 micrometers, and in some embodiments, from about 20 to about 60 micrometers.

### III. Composite Formation

To enhance the durability and stability, the first laminate is typically formed by directly extruding the film onto a surface of the nonwoven facing. This allows for an enhanced degree of contact between the film composition and fibers of the nonwoven facing, which further increases the ability of the nonwoven fibers to bond to the film composition. In this manner, a sufficient degree of bonding is achieved without requiring the application of a substantial amount of heat and pressure used in conventional calender bonding processes, which can damage the low strength nonwoven facing. If desired, lamination may be facilitated through the use of a variety of techniques, such as adhesives, suctional forces, and so forth. In one embodiment, for example, the film is biased toward the facing during lamination with a suctional force. Following lamination of the film to the nonwoven facing, an elastic polymer in the elastic film is cross-linked as described above.

The second facing/laminate may be formed using the same technique or a different technique depending on the desired application. Regardless, the first laminate and the second facing/laminate are positioned so that the films, if present, are in a face-to-face relationship. Through selective control over the polymer content and thickness, the films may be readily joined together under light pressure, even at ambient temperature conditions. Further, the films may be joined by thermal bonding, ultrasonic bonding, or adhesives.

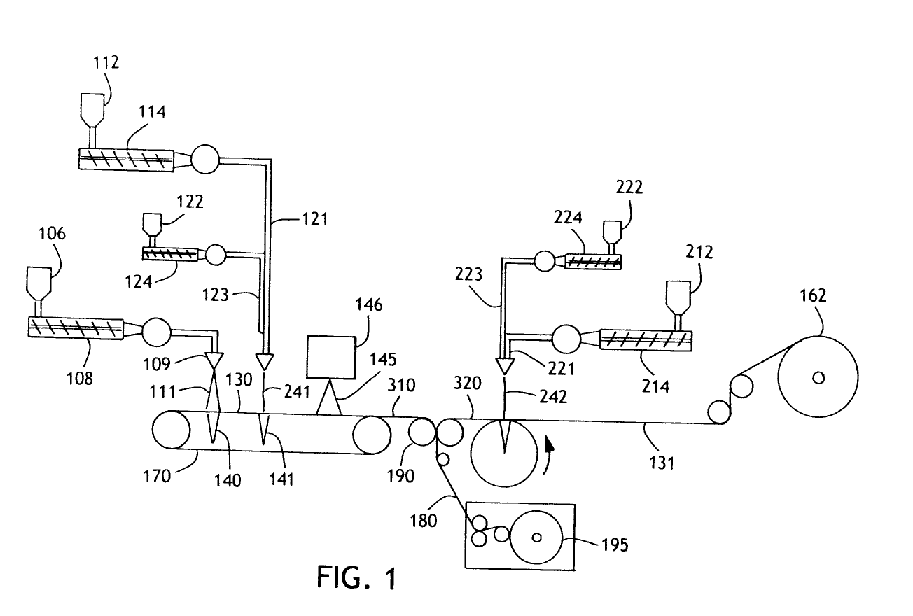

Various embodiments of the lamination technique of the present invention will now be described in greater detail. Referring to FIG. 1, for instance, one embodiment of a method for forming a composite is shown. In this embodiment, a first laminate 310 is formed from a meltblown facing 130 made in-line by feeding raw materials (e.g., polyethylene or polypropylene) into an extruder 108 from a hopper 106, and thereafter supplying the extruded composition to a meltblown die 109. As the polymer exits the die 109 at an orifice (not shown), high pressure fluid (e.g., heated air) attenuates and spreads the polymer stream into microfibers 111 that are randomly deposited onto a surface of a foraminous surface (e.g., wire, belt, fabric, and so forth) 170 to form a meltblown facing 130. A vacuum source 140 may aid in depositing the microfibers 111 on the foraminous surface 170 by drawing the high pressure fluid through the foraminous surface. It should be understood that the meltblown facing 130 may simply be unwound from a supply roll rather than formed in-line.

In the embodiment shown in FIG. 1, an elastic film is also formed that contains a single thermoplastic layer 123 and a single elastic layer 121. The raw materials of the elastic layer 121 may be added to a hopper 112 of an elastomeric extruder 114 and the raw materials of the thermoplastic layer 123 may be added to a hopper 122 of a thermoplastic extruder 124. The materials are dispersively mixed and compounded under at an elevated temperature within the extruders 114 and 124. The selection of an appropriate melt processing temperature will help melt and/soften the elastomeric polymer(s) of the film. The softened thermoplastic polymer(s) may then flow and become fused to the meltblown facing, thereby forming an integral laminate structure. Furthermore, because the thermoplastic polymer(s) may physically entrap or adhere to the fibers at the bond sites, adequate bond formation may be achieved without requiring substantial softening of the polymer(s) used to form the facing. Of course, it should be understood that the temperature of the facing may be above its softening point in certain embodiments.

Within the elastomeric extruder 114, for example, melt blending of the elastomeric composition may occur at a temperature of from about 50° C. to about 300° C., in some embodiments from about 60° C. to about 275° C., and in some embodiments, from about 70° C. to about 260° C. Melt blending of the thermoplastic composition may occur within the thermoplastic extruder 124 at a temperature that is the same, lower, or higher than employed for the elastomeric composition. For example, melt blending of the thermoplastic composition may in some instances occur at a temperature of from about 50° C. to about 250° C., in some embodiments from about 60° C. to about 225° C., and in some embodiments, from about 70° C. to about 200° C.

Any known technique may be used to form a film from the compounded material, including casting, flat die extruding, and so forth. In the particular embodiment of FIG. 1, for example, the elastic and thermoplastic layers are “cast” onto the meltblown facing 130, which is positioned on the foraminous surface 170, as is known in the art. A cast composite elastic film 241 is thus formed on the facing 130 such that the thermoplastic layer 121 is positioned directly adjacent to the facing 130. To enhance bonding between the composite elastic film 241 and the facing 130, a suctional force is applied to bias the composite elastic film 241 against an upper surface of the meltblown facing 130. This may be accomplished in a variety of ways (e.g., vacuum slots, shoes, rolls, and so forth) and at a variety of locations throughout the composite-forming process. In the embodiment shown in FIG. 1, for example, the foraminous surface 170 on which the composite elastic film 241 is cast is positioned above a vacuum source 141 capable of applying the desired suctional force. The amount of suctional force may be selectively controlled to enhance bonding without significantly deteriorating the integrity of the low strength facing. For example, pneumatic vacuum pressure may be employed to apply the suctional force that is about 0.25 kilopascals or more, in some embodiments about from about 0.3 to about 5 kilopascals, and in some embodiments, from about 0.5 to about 2 kilopascals. Such vacuum-assisted lamination allows for the formation of a strong composite without the need for a substantial amount of heat and pressure normally used in calender lamination methods that could otherwise diminish the integrity of the nonwoven facing.

After the composite elastic film 241 is laminated to the nonwoven facing 130, the elastic layer 121 is cross-linked by exposure to electromagnetic radiation 145 emanating from a cross-linking energy source 146. Crosslinking links polymer chains together to form a plurality of carbon-carbon covalent bonds. These bonds create a three-dimensional network from the original linear polymer chains. More specifically, crosslinking is induced by subjecting at least a portion of the elastic layer 121 to electromagnetic radiation, such as ultraviolet light, electron beam radiation, natural and artificial radio isotopes (e.g., α, β, and γ rays), x-rays, neutron beams, positively-charged beams, laser beams, and so forth. The actual dosage and/or energy level required may depend on the type of polymers and electromagnetic radiation. Specifically, the desired dosage and/or energy level may be adjusted to achieve the desired degree of crosslinking.

A second facing 320 is formed from a nonwoven facing 131 made in-line or originating from a supply roll (e.g., roll 162). The nonwoven facing 131 may include any nonwoven material, such as a meltblown web, spunbond web, bonded carded web and so forth. The second facing 320 also may contain a thermoplastic film 242 positioned adjacent to the nonwoven facing 131. The second facing 320 may include a laminate that is the same or similar in construction as the first laminate 310. In the illustrated embodiment, a vacuum lamination technique is employed. More specifically, the raw materials of an optional skin layer 221 are added to a hopper 212 of an extruder 214 and the raw materials of an optional core layer 223 are added to a hopper 222 of an extruder 224. The materials are then co-extruded onto the nonwoven facing 131 to form the thermoplastic film 242. A suctional force is also applied to bias the thermoplastic film 242 against an upper surface of the nonwoven facing 131 to form the second facing 320.

Once formed, the first laminate 310 and second facing 320 are then joined together to form a composite 180. Any of a variety of techniques may be employed to join the materials together. In the embodiment shown in FIG. 1, for example, the laminate 310 and facing 320 are joined together via a patterned bonding technique (e.g., point bonding, ultrasonic bonding, and so forth) in which the materials are supplied to a nip defined by at least one patterned roll (e.g., rolls 190). Point bonding, for instance, typically employs a nip formed between two rolls, at least one of which is patterned. Ultrasonic bonding, on the other hand, typically employs a nip formed between a sonic horn and a patterned roll. Regardless of the technique chosen, the patterned roll contains a plurality of bonding elements to concurrently bond the films of each laminate. The pressure exerted by the rolls (“nip pressure”) during pattern bonding may be relatively low and still achieve a considerable degree of peel strength. For example the nip pressure may range from about 1 to about 200 pounds per linear inch, in some embodiments from about 2 to about 100 pounds per linear inch, and in some embodiments, from about 5 to about 75 pounds per linear inch. The films of each laminate can readily bond together at relatively low temperatures. In fact, the rolls may even be kept at ambient temperature. For example, the rolls desirably have a surface temperature of from about 5° C. to about 60° C., in some embodiments from about 10° C. to about 55° C., and in some embodiments, from about 15° C. to about 50° C. Of course, it should be understood that higher nip pressures and/or temperatures may be employed if so desired. Also, the residence time of the materials may influence the particular bonding parameters employed.

Various processing and/or finishing steps known in the art, such as slitting, stretching, and so forth, may also be performed without departing from the spirit and scope of the invention. For instance, the composite may optionally be mechanically stretched in the cross-machine and/or machine directions to enhance extensibility. In the embodiment shown in FIG. 1, for example, the rolls 190 may possess grooves in the CD and/or MD directions that incrementally stretch the composite 180 in the CD and/or MD direction and also join together the laminates 310 and 320. Alternatively, the rolls 190 may join together the laminates 310 and 320 and separate grooved rolls (not shown) may be used to incrementally stretch the composite 180. Grooved satellite/anvil roll arrangements are described in U.S. Patent Application Publication Nos. 2004/0110442 to Rhim, et al. and 2006/0151914 to Gerndt, et al., which are incorporated herein in their entirety by reference thereto for all purposes.

Besides the above-described grooved rolls, other techniques may also be used to mechanically stretch the composite in one or more directions. For example, the composite may be passed through a tenter frame that stretches the composite. Such tenter frames are well known in the art and described, for instance, in U.S. Patent Application Publication No. 2004/0121687 to Morman, et al. The composite may also be necked. Suitable techniques necking techniques are described in U.S. Pat. Nos. 5,336,545, 5,226,992, 4,981,747 and 4,965,122 to Morman, as well as U.S. Patent Application Publication No. 2004/0121687 to Morman, et al., all of which are incorporated herein in their entirety by reference thereto for all purposes.

Referring again to FIG. 1, the composite 180, upon formation, may then be slit, wound, and stored on a take-up roll 195. The composite 180 may be allowed to retract in the machine direction prior to and/or during winding on to the take-up roll 195. This may be achieved by using a slower linear velocity for the roll 195. Alternatively, the composite 180 may be wound onto the roll 195 under tension.

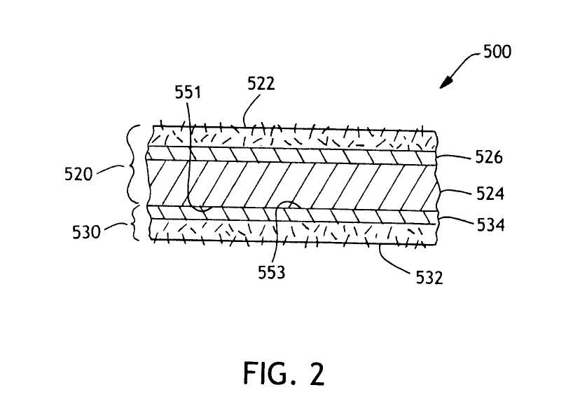

The resulting composite thus contains a cross-linked elastic film and optional thermoplastic skin layers positioned in a face-to-face relationship and bonded to each other. The first nonwoven facing is positioned in face-to-face relationship and bonded to either one side of the cross-linked elastic film or an optional skin layer. The second nonwoven facing is positioned in face-to-face relationship and bonded to either the other side of the cross-linked film or another optional skin layer. Referring to FIG. 2, for example, one embodiment of a composite 500 is shown that includes a first laminate 520 and a second laminate or nonwoven 530. The first laminate 520 is formed from a first nonwoven facing 522, cross-linked elastic film 524 optional skin layer 526. The second laminate 530 is formed from a second nonwoven facing 532 and an optional thermoplastic film or skin layer 534. In this embodiment, a lower surface 551 of the cross-linked elastic film 524 is positioned adjacent and bonded to an upper surface 553 of the thermoplastic film or skin layer 534. Such a composite structure provides a unique combination of strength and elastic properties in a cost-effective manner. For example, the use of a lightweight nonwoven facing enhances flexibility and reduces costs, while the use of separately cross-linked elastic films allows for the production of an effective elastic material without exposing the entire composite to the cross-linking process that could affect the integrity of the facings or films.

The resulting composite possesses a high degree of extensibility and elastic recovery. That is, the composite may exhibit an elongation at peak load (“peak elongations”) in the cross-machine direction, machine direction, or both of about 75% or more, in some embodiments about 100% or more, and in some embodiments, from about 150% to about 500%. The composite may also be elastic in that it is extensible in at least one direction upon application of the stretching force and, upon release of the stretching force, contracts/returns to approximately its original dimension. For example, a stretched material may have a stretched length that is at least 50% greater than its relaxed unstretched length, and which will recover to within at least 50% of its stretched length upon release of the stretching force. A hypothetical example would be a one (1) inch sample of a material that is stretchable to at least 1.50 inches and which, upon release of the stretching force, will recover to a length of not more than 1.25 inches. Desirably, the composite contracts or recovers at least 50%, and even more desirably, at least 80% of the stretched length.

The composite may also possess a high degree of strength in the machine direction and/or cross-machine direction. For example, the CD peak load of the composite may be at least about 1000 grams-force per inch (“gf/in”), in some embodiments from about 1100 to about 3000 gf/in, and in some embodiments, from about 1200 to about 2500 gf/in. Likewise, the MD peak load may be at least about 1500 grams-force per inch (“gf/in”), in some embodiments from about 1500 to about 6000 gf/in, and in some embodiments, from about 2000 to about 5000 gf/in.

### IV. Articles

The composite of the present invention may be used in a wide variety of applications. As noted above, for example, the composite may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Typically, absorbent articles include a substantially liquid-impermeable layer (e.g., outer cover), a liquid-permeable layer (e.g., bodyside liner, surge layer, and so forth), and an absorbent core. In one particular embodiment, the composite of the present invention may be used in providing elastic waist, leg cuff/gasketing, stretchable ear, side panel or stretchable outer cover applications.

Several examples of absorbent articles are described in U.S. Pat. No. 5,649,916 to DiPalma, et al.; U.S. Pat. No. 6,110,158 to Kielpikowski; U.S. Pat. No. 6,663,611 to Blaney, et al., which are incorporated herein in their entirety by reference thereto for all purposes. Further, other examples of personal care products that may incorporate such materials are training pants (such as in side panel materials) and feminine care products. By way of illustration only, training pants suitable for use with the present invention and various materials and methods for constructing the training pants are disclosed in U.S. Pat. No. 6,761,711 to Fletcher et al.; U.S. Pat. No. 4,940,464 to Van Gompel et al.; U.S. Pat. No. 5,766,389 to Brandon et al.; and U.S. Pat. No. 6,645,190 to Olson et al., which are incorporated herein in their entirety by reference thereto for all purposes.

The present invention may be better understood with reference to the following examples.

### Test Methods

### Hysteresis

The hysteresis of the elastic material was determined using a constant-rate-of-extension type of tensile tester. The tensile testing system was a Sintech Tensile Tester, which is available from MTS Corp. of Eden Prairie, Minn. The tensile tester was equipped with TESTWORKS 4.08B software from MTS Corporation to support the testing. An appropriate load cell was selected so that the tested value fell within the range of 10-90% of the full scale load. The elastomeric material was cut into strips, each having a width of three inches and a length of six inches. Both ends of the material were clamped into the opposing jaws of the apparatus, so that 2.5 centimeters of the length on each end of the material was maintained within the jaws and 10 centimeters of the length was available for stretching. The sample was held between a set of grips having a front and back face measuring 25.4 millimeters×76 millimeters. The grip faces were rubberized, and the longer dimension of the grip was perpendicular to the direction of pull. The grip pressure was pneumatically maintained at a pressure of 410 to 550 kilopascals. Each material strip was stretched at a rate of 51 centimeters per minute to a displacement of 10 centimeters while obtaining and recording the displacement and corresponding load values. The data was then reduced by integrating the area under the loading curve (representing force X displacement) and recorded as the “loading energy.” The material strip was then allowed to recover to a length where the stretching force is zero, again while obtaining and recording the displacement and corresponding load values. Area under the retraction curve was integrated and recorded as the “unloading energy.” Percentage hysteresis is determined according to the following equation:

$$\text{?} [ \frac{loading energy minus unloading energy}{loading energy} ] \times 100 \%$$

$$\text{?} \text{indicates text missing or illegible when filed}$$

### Tensile Properties:

The strip tensile strength values are determined in substantial accordance with AS™ Standard D-5034. Specifically, a sample is cut or otherwise provided with size dimensions that measure 25.4 millimeters (width)×152.4 millimeters (length). A constant-rate-of-extension type of tensile tester is employed, for example, a Sintech Tensile Tester, which is available from MTS Corp. of Eden Prairie, Minn. An appropriate load cell is selected so that the tested value falls within the range of 10-90% of the full scale load. The sample is held between grips having a front and back face measuring 25.4 millimeters×76 millimeters. The grip faces are rubberized, and the longer dimension of the grip is perpendicular to the direction of pull. The grip pressure is pneumatically maintained at a pressure of 410 to 550 kilopascals. The tensile test is run at a 51 centimeters per minute rate with a gauge length of 10 centimeters and a break sensitivity of 40%. Three samples are tested along the machine-direction (“MD”) and three samples are tested by along the cross direction (“CD”). In addition, the ultimate tensile strength (“peak load”), and peak elongation is recorded

### EXAMPLES

The ability to form an elastic composite from first and second nonwoven facings and a cross-linked elastic film was demonstrated. The first nonwoven facing was an 18 gram per square meter (gsm) meltblown web containing 100 wt. % DNDA 1082 NT-7 (Dow Chemical). DNDA 1082 NT-7 is a linear low density polyethylene resin with a melt index of 155 g/10 min (190° C., 2.16kg), a density of 0.933 g/cm³, and a melting point of 125° C. The meltblown web was formed on a forming belt using a 51 centimeter wide meltblown system having 12 capillaries per centimeter at a primary air temperature of 340° C. and a die temperature of 250° C. The forming belt speed was set to 18 meters per minute and the polymer throughput was approximately 3.3 grams per centimeter per minute, controlled by a metering pump.

Next, a film skin layer was then extruded onto the meltblown web and was suction forced and nipped onto the meltblown web while still the film was still molten. Vacuum pressures of 1″ H₂O to 15″H₂O were used for the suction application. The film skin layer was formed to a basis weight of 15 gsm using a 51 centimeter wide cast film die with the polymer hose and die temperature set to 230° C. The skin layer composition contained a blend of 96 weight percent polyethylene-based plastomer (EXACT™ 5361, ExxonMobil Chemical Company) and 4 weight percent a TiO2 concentrate (SCC-4857, Standridge Color Corporation). EXACT™ 5361 is a metallocene-catalyzed polyethylene plastomer having a density of 0.86 grams per cubic centimeter, a peak melting temperature of 36° C., and a melt index of 3.0 grams per 10 minutes (190° C., 2.16 kg).

Next, an elastomeric film core layer was extruded onto the film skin layer of the bi-layer laminate produced in the step above. A 51 centimeter wide cast film die was used and was set to 190° C. Four (4) different elastomeric film core formulations were used and are described in the Table 1 below.

TABLE 1

| | Primary | | |
|---|---|---|---|
| | Elastomer | Additives | |
| Sample # | Wt. Percent | Weight Percent | Basis Weight |
| 1 | 85% SBS | 14% Styron 666D | 31 gsm |
| | | 1% SCC-06SAM2184 | |
| 2 | 80% SBS | 10% Escorez 2203 | 27 gsm |
| | | 5% Affinity GA 1900 | |
| | | 4.5% SCC-23456 | |
| | | 0.5% SCC-22454 | |
| 3 | 80% D1160 | 20% Styron 666D | 36 gsm |
| 4 | 85% D1160 | 15% Escorene Blend | |

Samples 1 and 2 contained an SBS-polymer obtained from Dexco Polymers. Styron® 666D is a polystyrene polymer available from The Dow Chemical Company. Escorez™ 2203 is a tackifier available from ExxonMobil Chemical Company. Escorene™ blend refers to a 50/50 weight percent blend of Escorene™ 761.36 and 755.12, both EVA polymers available from ExxonMobil Chemical Company. Affinity™ GA 1900 is a polyethylene-based flow modifier available from The Dow Chemical Company. Kraton® D1160 is an SIS-based elastomer available from Kraton Polymers LLC. SCC-22454 is a compounded antioxidant agent, available from Standridge Color Corporation. SCC-23456 is a compounded antiblock agent, also available from Standridge Color Corporation. 06SAM2184 is a polymer processing aid, also available from Standridge Color Corporation.

The meltblown/film laminate was unwound at a speed of 4.6 meters per minute and the film side of the laminate was exposed to electron beam radiation using Advance Electron Beam's pilot line equipment operating at 150 KV accelerating voltage and 10 or 15 Megarads dosage.

After cross-linking, a second meltblown/film layer facing was prepared for lamination to the above described cross-linked elastic film/nonwoven laminate. A polypropylene-based 17 gsm thermally bonded carded web was used for the nonwoven material. A film layer, identical to the skin layer described above, was extruded onto the bonded carded web identically as described above for the first film laminate. The first and second nonwoven/film laminates were laminated together by positioning the films of each in face to face relationship and then passing the laminates between a pair of grooved rollers to form the final composite.

The effect of e-beam crosslinking on elastic properties was determined by measuring the hysteresis of the cross-linked films both before and after cross-linking. The material properties achieved for the composites are shown in Table 2 below.

TABLE 2
Material Properties

| | CD Hysteresis Before | CD Hysteresis Before |
|---|---|---|
| Sample # | Cross-Linking (Percent) | Cross-Linking (Percent) |
| 1 | sample broke | 11 |
| 2 | 20 | 15 |
| 3 | 24 | 11 |
| 4 | 17 | 15 |

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A method of forming an elastic composite, the method comprising:
extruding a thermoplastic composition directly onto a surface of a first nonwoven to form a first film, wherein the thermoplastic composition comprises a cross-linkable elastic polymer;
allowing the first film to bond to the first nonwoven to form a laminate;
cross-linking the cross-linkable elastic polymer; and
thereafter joining the first film directly to a second facing, the second facing comprising a second nonwoven.

2. The method of claim 1, wherein the second facing includes a second film, wherein the first film is positioned and bonded directly to the second film.

3. The method of claim 1, wherein the cross-linking step includes subjecting the cross-linkable elastic polymer to a dosage of electromagnetic radiation sufficient to crosslink the elastic polymer.

4. The method of claim 1, wherein the first nonwoven comprises polyethylene.

5. The method of claim 1, wherein the electromagnetic radiation has a wavelength of about 100 nanometers or less.

6. The method of claim 3, wherein the electromagnetic radiation is electron beam radiation.

7. The method of claim 6, wherein the dosage of the electromagnetic radiation is from about 1 to about 30 Megarads.

8. The method of claim 1, wherein the second nonwoven comprises polypropylene.

9. The method of claim 1, wherein the first nonwoven has a basis weight of about 45 grams per square meter or less and a peak load of about 350 grams-force per inch or less in the cross-machine direction.

10 The method of claim 1, wherein the first film includes an elastic layer and a thermoplastic layer, wherein the thermoplastic layer is positioned between the first nonwoven and the elastic layer.

11. The method of claim 10, wherein the elastic layer comprises the cross-linkable elastic polymer.

12 The method of claim 1, wherein the first nonwoven facing includes a meltblown web.

13. The method of claim 1, wherein the first and second laminates are passed through a nip formed between two rolls, wherein pressure is applied at the nip to bond the elastic film to the thermoplastic film.

14. The method of claim 13, wherein the rolls are grooved rolls.

15. An elastic composite comprising:
a first laminate that contains a first nonwoven facing and an elastic film, wherein the nonwoven facing has a basis weight of about 45 grams per square meter or less and a peak load of about 350 grams-force per inch or less in the cross-machine direction; and
a second laminate containing a second nonwoven facing and a thermoplastic film, wherein the first laminate and second laminate are joined together so that the elastic film is bonded and positioned adjacent to the thermoplastic film, and further wherein the elastic film is cross-linked by electromagnetic radiation and the first nonwoven facing has not been degraded by electromagnetic radiation.

16. The elastic composite of claim 15 where the second nonwoven facing has not been degraded by electromagnetic radiation.

17 The elastic composite of claim 15, wherein the first nonwoven facing is formed from a composition that contains a polyethylene polymer.

18. The elastic composite of claim 15, wherein the elastic film includes styrene-butadiene, styrene-isoprene, styrene-butadiene-styrene, styrene-isoprene-styrene, ethylene/α-olefin copolymer, or a combination thereof.

19. The elastic composite of claim 15, wherein the first nonwoven facing includes a meltblown web.

20. An absorbent article comprising the composite of claim 15.
